(19) United States
(12) Patent Application Publication
(10) Pub. No.: US 2008/0045917 A1
(43) Pub. Date: Feb. 21, 2008

(54) Stretchable outer cover for an absorbent article and process for making the same

(76) Inventors: Jean-Philippe Marie Autran, Wyoming, OH (US); Donald Carroll Roe, West Chester Township, OH (US); Terrill Allan Young, Colerain Township, OH (US); Joan Helen Mooney, Reading, OH (US); Fred NavaL Desai, Fairfield, OH (US); Bruno Johannes Ehrnsperger, Bad Soden am Taunus (DE); Andrew James Sauer, Colerain Township, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/810,801

(22) Filed: Jun. 7, 2007

Related U.S. Application Data
(60) Provisional application No. 60/811,580, filed on Jun. 7, 2006.

(51) Int. Cl. A61F 13/514; A61F 13/496

(52) U.S. Cl. 604/385.22; 604/385.29

(57) ABSTRACT

A stretchable outer cover for use with an absorbent article including an elastomeric film. The elastomeric film includes at least one skin layer that is less tacky than at least one core layer. The outer cover can include a nonwoven layer different structural combinations of spunbond fibers, meltblown fibers, and/or nanofibers. The combination of plastic and elastic components results in an outer cover that has favorable mechanical, physical, and aesthetic properties. The outer cover can be rendered either uniaxially or biaxially stretchable via a mechanical activation process.

### CROSS REFERENCE TO RELATED APPLICATIONS

This application claims the benefit of U.S. Provisional Application No. 60/811,580, filed Jun. 7, 2006, which is incorporated herein by reference.

### FIELD OF THE INVENTION

The invention provides at least one embodiment that generally relates to absorbent articles, and stretchable outer covers (“SOCs”) used therewith. More specifically, an embodiment of the invention relates to a stretchable outer cover having underwear-like, low-force, recoverable stretch. At least one embodiment of the invention also relates to an elastomeric film comprising an elastomeric core layer and an elastomeric skin layer, wherein the elastomeric skin layer has less tack than the elastomeric core layer.

### BACKGROUND OF THE INVENTION

Absorbent articles such as conventional taped diapers, pull-on diapers, training pants, incontinence briefs, and the like, offer the benefit of receiving and containing urine and/or other bodily exudates. Such absorbent articles can include a chassis that defines a waist opening and a pair of leg openings. A pair of barrier leg cuffs can extend from the chassis toward the wearer adjacent the leg openings, thereby forming a seal with the wearer's body to improve containment of liquids and other body exudates. Conventional chassis typically include an absorbent core that is disposed between a topsheet and a garment-facing outer cover (sometimes referred to as a backsheet).

The outer cover can include a stretchable waistband at one or both of its ends (e.g., proximal opposing laterally extending edges), stretchable leg bands surrounding the leg openings, and stretchable side panels, which additional components can be integral or separate discrete elements attached directly or indirectly to the outer cover. The remainder of the outer cover typically includes a non-stretchable nonwoven-breathable film laminate. Undesirably, however, these diapers sometimes do not conform well to the wearer's body in response to body movements (e.g., sitting, standing, and walking), due to the relative anatomic dimensional changes (which can, in some instances, be up to 50%) in the buttocks region caused by these movements. This conformity problem is further exacerbated because one diaper typically must fit many wearers of various shapes and sizes in a single product size.

Many of the elastomeric films used in absorbent articles have a relatively high tack, which may increase the difficulty of winding these films on rolls. Attempts to minimize the tack include laminating the tacky portion of the film to a nonwoven or include a non-tacky skin on the film prior to winding up on a roll. Typically, polyolefin skins are used. One disadvantage of using a skin is that it may negatively impact the elastomeric properties of the film. Activating the elastomeric film either by itself or after laminating it to one or more layers of nonwovens may generate pin holes due to the relatively high depth of engagement (“DOE”) needed to suitably break up the skin layer. Another disadvantage is that the non-elastic skin layer may add cost without providing any additional stretch.

Many caregivers and wearers prefer the look and feel of cotton underwear not provided by conventional diapers. For instance, cotton underwear includes elastic waist and leg bands that encircle the waist and leg regions of the wearer and provide the primary forces that keep the underwear on the wearer's body. Furthermore, the cotton outer cover (except in the waist and leg bands) can be stretched along the width and length directions in response to a relatively low force to accommodate the anatomic dimensional differences related to movement and different wearer positions. The stretched portion returns back to substantially its original dimension once the applied force is removed. In other words, the cotton outer cover of the underwear exhibits low-force, recoverable biaxial stretch that provides a conforming fit to a wider array of wearer sizes than conventional diapers.

Biaxially activation of the outer cover of an absorbent article may provide the low-force, recoverable stretch underwear-like material desired by some consumers, but the process for making such an outer cover may be difficult. Activating a typical outer cover in more than one direction may result in mechanical failure of the outer cover. These mechanical failings may manifest as pinholes, wrinkles or other functional or aesthetically undesirable features. In addition, providing a breathable outer cover for increased wearer comfort may also increase the difficulty of the manufacturing process due to the inclusion of apertures, micropores, and/or other discontinuities in the outer cover. Such opening may increase the possibility of mechanical failure of the outer cover materials during an activation process.

Accordingly, it would be desirable to provide an outer cover having an elastomeric skin layer with less tack than a core layer. It would further be desirable to provide a low-force, recoverable-stretch outer cover having the texture and aesthetics of cotton underwear. It would further be desirable to provide a process for manufacturing a breathable outer cover having the texture and aesthetics of cotton underwear.

### SUMMARY OF THE INVENTION

In order to provide a solution to the problems above at least one embodiment of the invention provides a stretchable outer cover for an absorbent article. The stretchable outer cover includes a multilayered elastomeric film layer. The multilayered elastomeric film layer includes at least one skin layer and at least one elastomeric core layer. The skin layer is elastomeric or plastoelastic. The elastomeric core layer includes a first elastomeric polypropylene. The skin layer is less tacky than the core layer.

### BRIEF DESCRIPTION OF THE DRAWINGS

FIG. 1 is cross section view of an absorbent article comprising an outer cover according to an embodiment of the invention.

FIG. 2 is cross section view of an outer cover according to an embodiment of the invention.

FIG. 3 is a scanning electron micrograph of a nonwoven substrate for use with an outer cover in an embodiment of the invention.

FIG. 4 is a graphical representation of the data listed in Table 9.

FIG. 5 is a graphical representation of the data listed in Table 10.

### DETAILED DESCRIPTION OF THE INVENTION

Definitions

As used herein, the following terms shall have the meaning specified thereafter:

The term “disposable,” as used herein in reference to absorbent articles, means that the absorbent articles are generally not intended to be laundered or otherwise restored or reused as absorbent articles (i.e., they are intended to be discarded after a single use and may be recycled, composted or otherwise discarded in an environmentally compatible manner).

The term “absorbent article” as used herein refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

The term “machine direction” (also “MD” or “length direction”) as applied to a film or nonwoven material, refers to the direction that is parallel to the direction of travel of the film or nonwoven as it is processed in the forming apparatus. The “cross machine direction” or “cross direction” (also “CD” or “width direction”) refers to the direction perpendicular to the machine direction and in the plane generally defined by the film or nonwoven material.

The term “longitudinal” as used herein refers to a direction running substantially perpendicular from a waist edge to an opposing waist edge of the article and generally parallel to the maximum linear dimension of the article. Directions within 45 degrees of the longitudinal direction are considered to be “longitudinal.”

The term “lateral” as used herein refers to a direction running from a longitudinal edge to an opposing longitudinal edge of the article and generally at a right angle to the longitudinal direction. Directions within 45 degrees of the lateral direction are considered to be “lateral.” The term “disposed” as used herein refers to an element being positioned in a particular place with regard to another element. When one group of fibers is disposed on a second group of fibers, the first and second groups of fibers generally form a layered, laminate structure in which at least some fibers from the first and second groups are in contact with each other. In some embodiments, individual fibers from the first and/or second group at the interface between the two groups can be dispersed among the fibers of the adjacent group, thereby forming an at least partially intermingled, entangled fibrous region between the two groups. When a polymeric layer (for example a film) is disposed on a surface (for example a group or layer of fibers), the polymeric layer can be laminated to or printed on the surface.

“Joined” refers to configurations whereby an element is directly secured to another element by affixing the element directly to the other element and to configurations whereby an element is indirectly secured to another element by affixing the element to intermediate member(s) which in turn are affixed to the other element.

As used herein, the term “stretchable” refers to materials which can stretch at least 5% on the upcurve of the Hysteresis Test at a load of 400 gf/cm. The term “non-stretchable” refers to materials which cannot stretch to at least 5% on the upcurve of the Hysteresis Test at a load of 400 gf/cm.

The terms “elastic” and “elastomeric” as used herein are synonymous and refer to any material that upon application of a biasing force, can stretch to an elongated length of at least 110% or even to 125% of its relaxed, original length (i.e., can stretch to 10% or even 25% more than its original length), without rupture or breakage. Further, upon release of the applied force, the material may recover at least 40%, at least 60%, or even at least 80% of its elongation. For example, a material that has an initial length of 100 mm can extend at least to 110 mm, and upon removal of the force would retract to a length of 106 mm (i.e., exhibiting a 40% recovery). The term “inelastic” refers herein to a material that cannot stretch to 10% more than its original length without rupture or breakage.

The terms “extensible” and “plastic” as used herein are synonymous and refer to any material that upon application of a biasing force, can stretch to an elongated length of at least 110% or even 125% of its relaxed, original length (i.e., can stretch to 10% or even 25% more than its original length), without rupture or breakage. Further, upon release of the applied force, the material shows little recovery, for example less than 40%, less than 20%, or even less than 10% of its elongation.

The terms “plastoelastic” and “elastoplastic” as used herein are synonymous and refer to any material that has the ability to stretch in a substantially plastic manner during an initial strain cycle (i.e., applying a tensile force to induce strain in the material, then removing the force allowing the material to relax), yet which exhibits substantially elastic behavior and recovery during subsequent strain cycles. Plastoelastic materials contain at least one plastic component and at least one elastic component, which components can be in the form of polymeric fibers, polymeric layers, and/or polymeric mixtures (including, for example, bi-component fibers and polymeric blends including the plastic and elastic components). Suitable plastoelastic materials and properties are described in U.S. 2005/0215963 and U.S. 2005/0215964.

As used herein, the term “activated” refers to a material which has been mechanically deformed so as to impart elastic extensibility to at least a portion the material, such as, for example by incremental stretching.

“Nanofibers” are sub-micron diameter fibers formed according to the process outlined in U.S. 2005/0070866 and U.S. 2006/0014460. Nanofibers generally have diameters of 0.1 μm to 1 μm, although larger diameters are possible. The number-average nanofiber diameter is generally in a range of 0.1 μm to 1 μm, for example 0.5 μm.

As used herein, the term “skin layer” generally refers to one or more layers in a multilayer film coextruded with at least one other layer (typically a core layer) such that each of the one or more skin layers represent less than 25%; or even less than 10% of the total film thickness. It is to be understood that when multiple skin layers are present the thickness of each skin layer need not necessarily be the same.

As used herein, the term “core layer” generally refers to one or more layers in a multilayer film coextruded with at least one other layer (typically a skin layer) such that each of the one or more core layers represent more than 50%; or even more than 75% of the total film thickness. It is to be understood that when multiple core layers are present the thickness of each core layer need not necessarily be the same.

As used herein, the term “underwear-like” generally refers to a substrate that exhibits low-force, recoverable stretch, which it similar to typical the characteristics exhibited by the cotton fabric portion of cotton underwear (this excludes the waist band and leg bands portions). For example, a substrate such as an outer cover for an absorbent article, that exhibits a load at 15% strain of less than 40 g/cm is considered underwear-like.

As used herein, “extrusion-lamination” generally means a process where a polymer is extruded onto at least one other nonwoven, and while still in a partially molten state, bonds to one side of the nonwoven, or by depositing onto an extruded molten polymer, a nonwoven.

### General Description of the Embodiments

The stretchable outer covers (“SOCs”) according to at least one embodiment of the invention may include at least one elastic material and at least one plastic material. The stretchable outer cover (“SOC”) may include a layer of polymeric material and a nonwoven layer disposed on the polymeric material. The nonwoven material and the polymeric layer can be formed (independently) from a plastoelastic material, an elastic material, or a plastic material. Although the SOC may have at least one plastic material and at least one elastic material, the two components can be included in the SOC in the form of a single plastoelastic material.

In certain embodiments of the invention, the SOC may include a polymeric layer in the form of a polymeric film laminated to the nonwoven material. These embodiments may have three additional aspects in which: (1) a layer of plastoelastic nonwoven material is laminated to a plastic polymeric film, (2) a layer of plastoelastic nonwoven material is laminated to a plastoelastic polymeric film, and (3) a layer of plastic nonwoven material is laminated to a plastoelastic polymeric film. When both the nonwoven material and the polymeric film are formed from a plastoelastic material, they can be formed from either the same or different plastoelastic materials. In certain embodiments, the SOC may include a layer of nonwoven material, such as, for example a layer of plastic fibers, onto which an elastomeric layer is printed or laminated in the form of a pattern or film.

The SOC of at least one embodiment of the invention has low-force, recoverable stretch, similar to the fabric of cotton underwear. In some embodiments, the outer cover may have a low force at a specific elongation. Since the outer cover can have different stretch properties in different directions, stretch properties may be measured in the longitudinal direction (machine direction) and in the lateral direction (cross machine direction). In some embodiments, at 15% strain, the outer cover may have a first cycle load less than 40 g/cm; 30 g/cm; 20 g/cm; or even less than 15 g/cm. In some embodiments, at 50% strain, the outer cover may have a first cycle load less than 100 g/cm; 75 g/cm; 40 g/cm or even less than 30 g/cm. Additionally, in some embodiments, the outer cover may also have a percentage set that is less than 40%; 30%; 20% or even less than 10%. It is believed that an outer cover with such properties may be more underwear-like.

In certain embodiments, an outer cover according to at least one embodiment of the invention may comprise an elastomeric film that is laminated to at least one non-elastic nonwoven. Each layer of nonwoven may have a basis weight of less than 50 g/m²; between 10 and 30 g/m²; or even between 10 and 20 g/m². The basis weight of the elastomeric film may be less than 40 g/m²; 30 g/m²; 25 g/m²; or even less than 15 g/m².

Since, the elastomer included in an absorbent article may be one of the more expensive components of the diaper, and since the area of the outer cover, hence elastomer usage, may be large for an all-over stretch outer cover, it may be desirable to be able to commercially make an outer cover with a low basis weight elastomer that is relatively inexpensive. Elastomeric polypropylenes may be attractive candidates, e.g., VISTAMAXX from Exxon-Mobil, as they are typically less expensive than conventional elastomers such as styrenic block copolymers. In addition, it may be easier to extrude these elastomeric polypropylenes at low basis weights (e.g., 10-40 g/m²) commercially compared to the styrenic block polymers, due to their higher melt strengths. Finally, since many other absorbent article components are often made of polypropylene, mechanical bonding with the elastomeric polypropylenes may be easier.

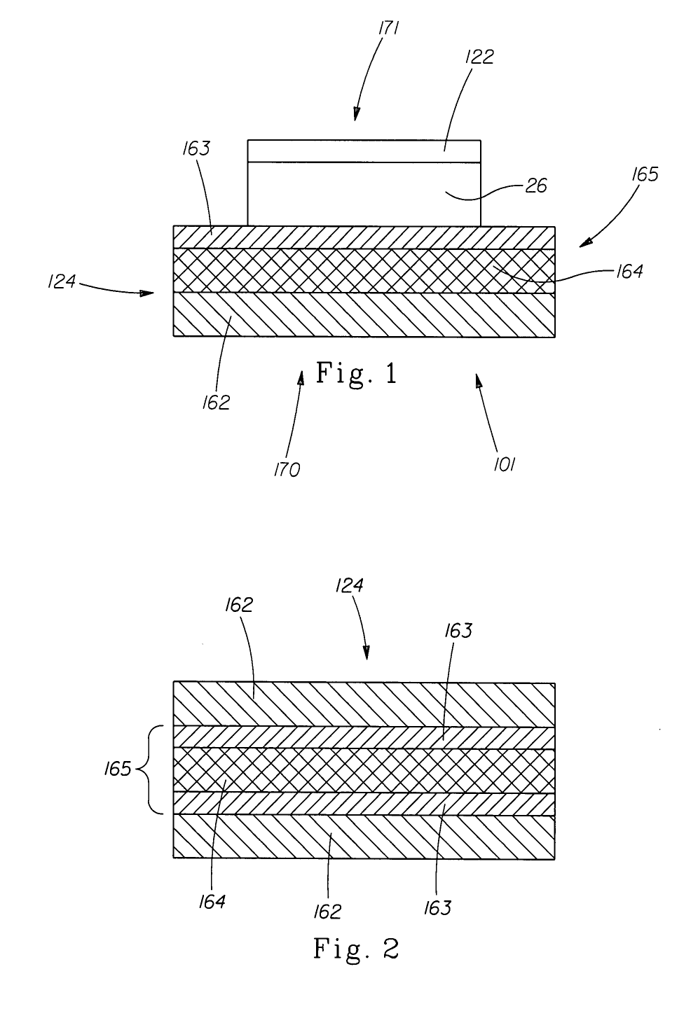

FIG. 1 shows a schematic view of an example of an absorbent article 101 that includes an outer cover 124 according to at least one embodiment of the invention. In this example, the outer cover 124 is a bilaminate formed from an elastomeric film 165 and a nonwoven 162. The outer cover 124 has a body facing side 171 and a garment facing side 170. In addition to an outer cover 124, the absorbent article may also include a topsheet 122 joined to the absorbent core 26 or any other component by any means commonly known in the art, such as, for example adhesive. The absorbent core 26 may be joined to the outer cover 124. The outer cover 124 shown in FIG. 1 may include an elastomeric film 165 comprising a skin layer 163 and a core layer 164. The skin layer 163 may be joined to the core layer 164 in a face to face configuration to form a laminate. In a film-nonwoven bilaminate, the skin layer 163 is generally disposed on the body facing side 171 of the outer cover 124. While only a single skin layer 163 and a single core layer 164 is shown in FIG. 1, it is to be understood that the outer cover 124 may include additional skin and/or core layers, as desired. Optionally, the outer cover 124 may also include a second nonwoven material 162 as shown in FIG. 2. In FIG. 2, the elastomeric film 165 has two skin layers 163 and two nonwoven layers 162. Such a structure may be formed when the steps of film formation and lamination to nonwovens are done at different times and/or locations. The nonwoven 162 may be joined to the elastomeric film 165 by any means commonly known in the art

Like underwear, the absorbent article may also include elastic waist and leg bands in addition to the Stretchable Outer Cover (SOC). These bands ideally would cover substantially the entire circumference around the waist and legs. These waist and leg bands help decrease diaper sag, especially since the SOC offers only little return force. These waist and leg bands would be laminates of an elastic material and at least one nonwoven, wherein the elastic is prestretched prior to bonding it to the nonwoven (i.e., Stretch Bonded Laminate). The elastic material could be in the form of strands or film or a nonwoven. Any bonding technique known in the industry can be used to bond the elastic material to the nonwoven. Some examples are adhesive bonding, ultrasonic bonding, thermal point bonding, mechanical bonding with pressure and/or heat, and the like.

The elastic waist and leg bands are 5 to 40 mm wide. One example is a trilaminate comprising Spandex strands, having a decitex of 400 to 1500, and laminated to two layers of nonwovens. These strands, which run along the machine direction of the web, are prestretched to 100-300% prior to laminating to the nonwoven. The waist and leg bands are next prestretched prior to bonding them to the SOC.

Polymeric Materials

The plastoelastic materials according to at least one embodiment of the invention, whether included in a nonwoven fibrous layer or a polymeric film layer, may include an elastomeric component and a plastic component. The components can be in the form of fibers (e.g., elastomeric fibers, plastic fibers), in the form of a multilayer film (e.g., an elastomeric layer, a plastic layer), or as an element of a polymeric mixture (e.g., bi-component fibers, plastoelastic blend fibers, a plastoelastic blend layer). One plastoelastic material can be in the form of a plastoelastic blend of an elastomeric component and a plastic component. The plastoelastic blend can form either a heterogeneous or a homogeneous polymeric mixture, depending upon the degree of miscibility of the elastomeric and plastic components. For heterogeneous mixtures, the resultant stress-strain properties of the plastoelastic material may be improved when micro-scale dispersion of any immiscible components is achieved (i.e., any discernable discrete domains of pure elastomeric component or pure plastic component have an equivalent diameter less than 10 microns). Suitable blending means are known in the art and include a twin screw extruder (e.g., POLYLAB twin screw extruder, available from Thermo Electron, Karlsruhe, Germany). If the plastoelastic blend forms a heterogeneous mixture, one component can form a continuous phase that encloses dispersed particles of the other component. Another example of a plastoelastic material includes plastoelastic bi-component fibers, in which a single fiber has discrete regions of the elastomeric and plastic components in, for example, a core-sheath (or, equivalently, a core-shell) or a side-by-side arrangement. Another example of a plastoelastic material includes mixed fibers, in which some fibers are formed essentially entirely from the elastomeric component and the remaining fibers are formed essentially entirely from the plastic component. Polymeric materials can also include combinations of the foregoing (e.g., plastoelastic blend fibers and bicomponent fibers, plastoelastic blend fibers and mixed fibers, bicomponent fibers and mixed fibers). A further example of a plastoelastic material is a plastoelastic blend in the form of a heterogeneous mixture having a co-continuous morphology with both phases forming interpenetrating networks.

Suitable examples of plastoelastic materials include the elastomeric component in a range of 5 wt. % to 95 wt. % and from 40 wt. % to 90 wt. %, based on the total weight of the plastoelastic material. Suitable examples of the plastoelastic materials include the plastic component in a range of 5 wt. % to 95 wt. %, and from 10 wt. % to 60 wt. %, based on the total weight of the plastoelastic material. When the plastoelastic material includes mixed elastic and plastic fibers, the elastic fibers may be included in an amount from 40 wt. % to 60 wt. %, for example 50 wt. % (with the approximate balance being the plastic fibers), based on the total weight of the mixed elastic and plastic fibers. When the plastoelastic material includes bi-component fibers, the plastic component (e.g., in the form of a sheath) may be included in an amount of 20 wt. % or less or 15 wt. % or less, for example 5 wt. % to 10 wt. % (with the approximate balance being the elastic component, for example as a fiber core), based on the total weight of the bi-component fibers. When the plastoelastic material includes a plastoelastic blend, the elastic component may be included in an amount from 60 wt. % to 80 wt. %, for example 70 wt. % (with the approximate balance being the plastic component), based on the total weight of the plastoelastic blend. In some embodiments, the plastoelastic material can include more than one elastomeric component and/or more than one plastic component, in which case the stated concentration ranges apply to the sum of the appropriate components and each component may be incorporated at a level of at least 5 wt. %.

The elastomeric component may provide the desired amount and force of recovery upon the relaxation of an elongating tension on the plastoelastic material, especially upon strain cycles following the initial shaping strain cycle. Many elastic materials are known in the art, including synthetic or natural rubbers, thermoplastic elastomers based on multi-block copolymers, such as those comprising copolymerized rubber elastomeric blocks with polystyrene blocks, thermoplastic elastomers based on polyurethanes (which form a hard phase that provides high mechanical integrity when dispersed in an elastomeric phase by anchoring the polymer chains together), polyesters, polyether amides, elastomeric polyethylenes, elastomeric polypropylenes, and combinations thereof. Some particularly suitable examples of elastic components include styrenic block copolymers, elastomeric polyolefins, and polyurethanes.

Other particularly suitable examples of elastic components include elastomeric polypropylenes. In these materials, propylene represents the majority component of the polymeric backbone, and as a result, any residual crystallinity possesses the characteristics of polypropylene crystals. Residual crystalline entities embedded in the propylene-based elastomeric molecular network may function as physical crosslinks, providing polymeric chain anchoring capabilities that improve the mechanical properties of the elastic network, such as high recovery, low set and low force relaxation. Suitable examples of elastomeric polypropylenes include an elastic random poly(propylene/olefin) copolymer, an isotactic polypropylene containing stereoerrors, an isotactic/atactic polypropylene block copolymer, an isotactic polypropylene/random poly(propylene/olefin) copolymer block copolymer, a stereoblock elastomeric polypropylene, a syndiotactic polypropylene block poly(ethylene-co-propylene) block syndiotactic polypropylene triblock copolymer, an isotactic polypropylene block regioirregular polypropylene block isotactic polypropylene triblock copolymer, a polyethylene random (ethylene/olefin) copolymer block copolymer, a reactor blend polypropylene, a very low density polypropylene (or, equivalently, ultra low density polypropylene), a metallocene polypropylene, and combinations thereof. Suitable polypropylene polymers including crystalline isotactic blocks and amorphous atactic blocks are described, for example, in U.S. Pat. Nos. 6,559,262, 6,518,378, and 6,169,151. Suitable isotactic polypropylene with stereoerrors along the polymer chain are described in U.S. Pat. No. 6,555,643 and EP 1 256 594 A1. Suitable examples include elastomeric random copolymers (RCPs) including propylene with a low level comonomer (e.g., ethylene or a higher a-olefin) incorporated into the backbone. Suitable elastomeric RCP materials are available under the names VISTAMAXX (available from ExxonMobil, Houston, Tex.) and VERSIFY (available from Dow Chemical, Midland, Mich.). When the SOC includes a printed elastic material, the elastomeric component may be a styrenic block copolymer.

The plastic component of the plastoelastic material may provide the desired amount of permanent plastic deformation imparted to the material during the initial shaping strain cycle, whether included in a plastoelastic blend or in a discrete plastic component. Typically, the higher the concentration of a plastic component in the plastoelastic material, the greater the possible permanent set following relaxation of an initial straining force on the material. Suitable plastic components generally include higher crystallinity polyolefins that are plastically deformable when subjected to a tensile force in one or more directions, for example high density polyethylene, linear low density polyethylene, very low density polyethylene, a polypropylene homopolymer, a plastic random poly(propylene/olefin) copolymer, syndiotactic polypropylene, polybutene, an impact copolymer, a polyolefin wax, and combinations thereof Another suitable plastic component is a polyolefin wax, including microcrystalline waxes, low molecular weight polyethylene waxes, and polypropylene waxes. Suitable materials include LL6201 (linear low density polyethylene; available from ExxonMobil, Houston, Tex.), PARVAN 1580 (low molecular weight polyethylene wax; available from ExxonMobil, Houston, Tex.), MULTIWAX W-835 (microcrystalline wax; available from Crompton Corporation, Middlebury, Conn.); Refined Wax 128 (low melting refined petroleum wax; available from Chevron Texaco Global Lubricants, San Ramon, Calif.), A-C 617 and A-C 735 (low molecular weight polyethylene waxes; available from Honeywell Specialty Wax and Additives, Morristown, N.J.), and LICOWAX PP230 (low molecular weight polypropylene wax; available from Clariant, Pigments & Additives Division, Coventry, R.I.).

Other polymers suitable as the plastic component, whether included in the nonwoven fibers or the polymeric layer, are not particularly limited as long as they have plastic deformation properties. Suitable plastic polymers include polyolefins generally, polyethylene, linear low density polyethylene, polypropylene, ethylene vinyl acetate, ethylene ethyl acrylate, ethylene acrylic acid, ethylene methyl acrylate, ethylene butyl acrylate, polyurethane, poly(ether-ester) block copolymers, poly(amide-ether) block copolymers, and combinations thereof. Suitable polyolefins generally include those supplied from ExxonMobil (Houston, Tex.), Dow Chemical (Midland, Mich.), Basell Polyolefins (Elkton, Md.), and Mitsui USA (New York, N.Y.). Suitable plastic polyethylene films are available from RKW US, Inc. (Rome, Ga.) and from Cloplay Plastic Products (Mason, Ohio).

Fibrous Materials

The nonwoven fibrous material according to at least one embodiment of the invention is generally formed from fibers which are interlaid in an irregular fashion using such processes as meltblowing, spunbonding, spunbonding-meltblowing-spunbonding (SMS), air laying, coforming, and carding. The nonwoven material may include spunbond fibers. The fibers of the nonwoven material may be bonded together using conventional techniques, such as thermal point bonding, ultrasonic point bonding, adhesive pattern bonding, and adhesive spray bonding. The basis weight of the resulting nonwoven material can be as high as 100 g/m², but may also be less than 80 g/m², less than 60 g/m², and even less than 50 g/m², for example less than 40 g/m². Unless otherwise noted, basis weights disclosed herein are determined using European Disposables and Nonwovens Association (“EDANA”) method 40.3-90.

In one example of an embodiment of the invention, the nonwoven material can include two or, optionally, three different layers of fibers: a first layer of nonwoven fibers having a first number-average fiber diameter, a second layer of fibers having a second number-average fiber diameter that is smaller than the first number-average fiber diameter, and optionally a third layer of fibers having a third number-average fiber diameter that is smaller than the second number-average fiber diameter. The ratio of the first diameter to the second diameter is generally 2 to 50, or 3 to 10, for example 5. The ratio of the second diameter to the third diameter is generally 2 to 10, for example 5. In this embodiment, the second layer of fibers is disposed on the first layer of nonwoven fibers, and the third layer of fibers (when included) is disposed on the second layer of fibers. This arrangement can include the case where the first and second (and optionally third) fiber layers form essentially adjacent layers such that a portion of the layers overlap to form an interpenetrating fiber network at the interface (e.g., fibers from the first and second layers overlap and/or fibers from the second and third layers overlap). This arrangement can also include the case where the first and second fiber layers are essentially completely intermingled to form a single heterogeneous layer of interpenetrating fibers.

In this example of an embodiment, the first number-average fiber diameter may be in a range of 10 μm to 30 μm, for example 15 μm to 25 μm. Suitable fibers for the first group of nonwoven fibers include spunbond fibers. The spunbond fibers can include the various combinations of elastomeric and plastic components described above.

In this example of an embodiment, the second number-average fiber diameter may be in a range of 1 μm to 10 μm, for example 1 μm to 5 μm. Suitable fibers for the second group of fibers include meltblown fibers, which can be incorporated into the nonwoven material in one or more layers. The meltblown fibers may have a basis weight in a range of 1 g/m² to 20 g/m² or 4 g/m² to 15 g/m², distributed among the various meltblown layers. The meltblown fibers can include the various combinations of elastomeric and plastic components described above, and may also include elastic materials and/or plastoelastic materials. A higher elastomeric content may be preferred when higher depths of activation are required and/or when lower permanent set values in the outer cover are desired. Elastomeric and plastic polyolefin combinations can be utilized to optimize the cost/performance balance. In some embodiments, the elastomeric component can include a very low crystallinity polypropylene (e.g., VISTAMAXX polypropylene available from ExxonMobil, Houston, Tex.). In certain embodiments of the invention, the elastomeric nonwoven may include at least one spunbond layer comprising elastic fibers and at least one layer of meltblown fibers comprising elastic, plastoelastic or plastic fibers.

The fine fibers of the meltblown layer may enhance the opacity of the SOC, which is typically a desirable feature in outer covers. The meltblown fibers may also have the beneficial effect of improving the structural integrity of the nonwoven material when the meltblown fibers overlap and are dispersed among the other nonwoven fibers of the nonwoven material, for example in an SMS nonwoven laminate in which the meltblown layer is disposed between and joined to two spunbond layers. The self-entanglement resulting from the incorporation of fibers having substantially different length scales can increase the internal adhesive integrity of the nonwoven material, thereby lessening (and potentially even eliminating) the need for the bonding of the nonwoven material. The meltblown fibers can also form a “tie-layer” increasing the adhesion between the other nonwoven fibers and an adjacent polymeric layer, in particular when the meltblown fibers are formed from an adhesive material. The presence of the meltblown fibers can also have the beneficial effect of reducing the post-activation % set by a relative amount of at least 5% (i.e., relative to a nonwoven material that is otherwise the same except for the meltblown fibers) or at least 8%, for example at least 10%.

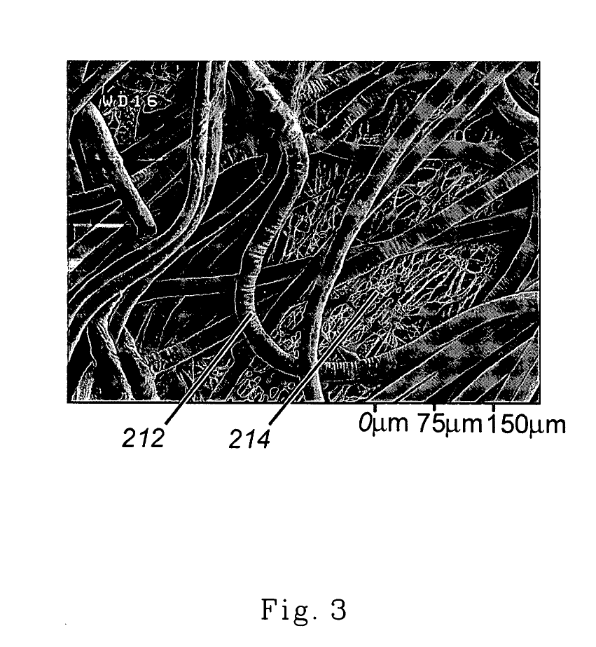

The second number-average fiber diameter may alternatively or additionally be in a range of 0.1 μm to 1 μm, for example 0.5 μm. Suitable fibers for such a second group of fibers include nanofibers, which can have the compositions described above for meltblown fibers. Using nanofibers either in place of meltblown fibers (in which case the nanofibers form the second layer of fibers) or in addition to meltblown fibers (in which case the nanofibers form the third layer of fibers) can further increase the opacity of the outer cover, and can also provide the structural and adhesive advantages mentioned above in relation to meltblown fibers. FIG. 3 illustrates a layer of finer nanofibers 214 below a layer of coarser spunbond fibers 212 in an SEM of a spunbond-nanofiber-spunbond (“SNS”) laminate. From FIG. 3, it is apparent that the void surface areas resulting in the upper spunbond layer are substantially filled by the underlying nanofiber layer, thereby improving the opacity. When they are included, the nanofibers may have a basis weight in a range of 1 g/m² to 7 g/m², for example in a range of 3 g/m² to 5 g/m². At such levels, the nanofibers can provide a relative increase (i.e., relative to a nonwoven material that is otherwise the same except for the nanofibers) in the opacity of the nonwoven material of at least 5%, or at least 8%, for example at least 10%. In an alternate embodiment, opacifying particles such as titanium dioxide can be included in the nanofibers to further increase the opacity. In certain embodiments, the elastomeric nonwoven may comprise at least one spunbond layer comprising elastic fibers and at least one layer of nanofibers comprising elastic, plastoelastic and/or plastic fibers.

When nanofibers are included in the nonwoven layer of an outer cover according to at least embodiment of the invention it may be possible to increase the opacity of the outer cover. For example, in order to provide an outer cover having an opacity of 65%, as measured according to the opacity test, the basis weight of a typical meltblown layer may need to be 8 g/m²; and for 70% opacity, the basis weight may need to be over 10 g/m². With nanofibers, however, in order to achieve an opacity of 65%, the basis weight of the nanofibers may be 3 g/m²; and for 70% opacity, the basis weight may be 5 g/m².

In another example of an embodiment of the invention, the nonwoven material may include at least four, and optionally five, layers of fibers of differing kinds in a stacked arrangement. The first (top) layer may include spunbond fibers, such as, for example a plastoelastic material that includes but is not limited to mixed elastomeric fibers and plastic fibers, bi-component elastomeric and plastic fibers, and plastoelastic blend fibers; including elastomeric polypropylene. The second layer may be disposed on the first layer and can include meltblown fibers, such as, for example elastomeric fibers that include but are not limited to elastomeric polypropylene or elastomeric polyethylene. The third layer may be disposed on the second layer and can include nanofibers that are generally either elastomeric fibers (for example including either elastomeric polypropylene or elastomeric polyethylene) or plastoelastic blend fibers (for example including elastomeric polypropylene). The fourth layer may be disposed on the third layer and can include meltblown fibers, such as, for example plastoelastic blend fibers, including elastomeric polypropylene. Other possible materials for the first through fourth layers are the same as those described above under “Polymeric Materials.”

The optional fifth (bottom) layer may be joined to the fourth layer and can includes spunbond (or, alternatively, carded) fibers that are generally either plastic fibers (for example including high-extensibility nonwoven fibers or a high-elongation carded web material) or plastoelastic blend fibers. When the fifth layer includes plastic fibers, it may be advantageous to provide plastic fibers that are extensible enough to survive the mechanical activation process. Suitable examples of such sufficiently deformable spunbond fibers are disclosed in WO 2005/073308 and WO 2005/073309. Suitable commercial plastic fibers for the fifth layer include a deep-activation polypropylene, a high-extensibility polyethylene, and polyethylene/poly-propylene bi-component fibers (all available from BBA Fiberweb Inc., Simpsonville, S.C.). The fifth layer can be added to the nonwoven material at the same time as the first four layers, or the fifth layer can be added later in a production process for an absorbent article. Adding the fifth layer later in the production process permits greater SOC flexibility, for example allowing the intercalation of absorbent article components (e.g., a high-performance elastomeric band) into the SOC and permitting the omission of the fifth layer in regions where it is not required in the absorbent article (e.g., where the SOC is positioned on the absorbent core).

In various embodiments of the invention, the coarse spunbond fibers may provide the desirable mechanical properties of the resulting material, the fine meltblown fibers may increase the opacity and the internal adhesive integrity of the resulting material, and the even finer nanofibers may further increase the opacity. Each spunbond or carded layer may be included in the nonwoven material at a basis weight of at least 10 g/m², for example at least 13 g/m² and may be included in the nonwoven material at a basis weight preferably of 50 g/m² or less, for example 30 g/m² or less. Each meltblown and nanofiber layer may be included in the nonwoven material at a basis weight of at least 1 g/m², for example at least 3 g/m². The final nonwoven material has a basis weight in a range of 25 g/m² to 100 g/m², for example 35 g/m² to 80 g/m². The final outer cover can also include a laminated polymeric film or a printed elastic layer of the kinds described below.

For SOCs including an elastomeric film and plastic nonwovens, pin holing can be a potential issue during mechanical activation, especially at high speeds. In some embodiments of the invention it is critical to prevent pinholing during activation. Extensible nonwovens may help mitigate or even resolve this issue. A key property that characterizes an extensible nonwoven is its peak elongation (i.e., the higher the peak elongation, the more extensible the nonwoven). Tearing of the SOC may result during mechanical activation when including conventional plastic nonwovens in the SOC. On the other hand, plastic nonwovens that have peak elongations greater than 100%, greater than 120%, or even greater than 150%, for example 180%. may reduce the likelihood of tearing the SOC during mechanical activation. One suitable example of such an extensible nonwoven is Softspan 200 made by BBA (Fiberweb), Simpsonville, SC, which has a peak elongation of 200%.

Laminated Polymeric Films and Printed Elastic Layers

The polymeric film according to at least one embodiment of the invention can be formed with conventional equipment and processes, such as, for example using cast film or blown film equipment. The polymeric film also can be coextruded with the nonwoven fibers. The polymeric film also can be colored, for example by adding a dye to the resin before the film is formed (which method of coloration can also be used for the polymeric fibrous materials of the invention). The basis weight of the resulting polymeric film may in a range of 10 g/m² to 40 g/m² or in a range of 12 g/m² to 30 g/m², for example in a range of 15 g/m² to 25 g/m². The polymeric film may have a thickness of less than 100 μm or the polymeric film may have a thickness of 10 μm to 50 μm.

In certain embodiments, the polymeric film may be formed from multiple layers coextruded into a single multi-layer film. A multi-layer film may permit tailoring the properties of the film to the specific needs of the application by decoupling the bulk and surface properties in the final film. For instance, antiblock additives may be included in greater weight percent to the skin layers (i.e., an exterior layer in the final film) than the core layers. The skin layers may include up to 2 weight % antiblocking by weight of the skin layer composition while the core layer includes only 0.2 weight % by weight of the core layer composition or even no antiblocking additive. In certain embodiments, a higher crystallinity, higher melting-point elastomeric component (e.g., VM3000 film-grade VISTAMAXX, having a first melting temperature T_{m,l}>60° C., instead of VM1100 film-grade VISTAMAXX, having a first melting temperature Tm_{m,l}˜50° C.) may be used in the skin layer to reduce tackiness. A plastoelastic skin layer can similarly reduce tackiness. Both tackiness-reduction options can enhance the thermal stability of the final film and increase its toughness, thereby preventing tear initiation and/or propagation in apertured films and laminates. It may be desirable to ensure that the amount of tack in the skin layer is low enough to enable unwinding of the film from a roll.

The core layer (i.e., an interior layer in the final film) can include blends of elastomeric polypropylene and a styrenic block copolymer. Alternatively or additionally, both the core and skin layers can contain sufficient amounts of filler particles to become microporous upon activation (thereby increasing the breathability of the film), yet they can have different base polymeric components. Three examples of suitable multi-layer films include: (1) a lower melting point elastomeric polypropylene core laminated with a higher melting point elastomeric polypropylene skin, (2) a lower melting point blended core of elastomeric polypropylene and a styrenic block copolymer laminated with a higher melting point elastomeric polypropylene skin, and (3) a filled blended core of a plastoelastic polymer and a styrenic block copolymer laminated with a filled plastic polyethylene skin.

The elastomeric component can be printed onto the plastic layer of nonwoven fibers as a continuous film or as a pattern. If printed as a pattern, the pattern can be relatively regular, covering substantially the entire area of the outer cover, for example, in a continuous mesh pattern or a discontinuous dot pattern. The pattern can also include regions of relatively higher or lower basis weights wherein the elastomeric component has been applied onto at least one region of the plastic layer of nonwoven fibers to provide particular stretch properties to a targeted region of the SOC (i.e., after biaxial mechanical activation).

The polymeric film can optionally include organic and inorganic filler particles. The filler particles may be small (e.g., 0.4 μm to 8 μm average diameter) to produce micropores that are sufficient to simultaneously promote the breathability of the film and maintain the liquid water barrier properties of the film. Examples of suitable fillers include calcium carbonate, non-swellable clays, silica, alumina, barium sulfate, sodium carbonate, talc, magnesium sulfate, titanium dioxide, zeolites, aluminum sulfate, cellulose-type powders, diatomaceous earth, magnesium sulfate, magnesium carbonate, barium carbonate, kaolin, mica, carbon, calcium oxide, magnesium oxide, aluminum hydroxide, glass particles, pulp powder, wood powder, chitin, chitin derivatives, and polymer particles. A suitable inorganic filler particle for improving the breathability of the film is calcium carbonate. Suitable organic filler particles include submicron (e.g., 0.4 μm to 1 μm) polyolefin crystals that are formed by the crystallization of the low crystallinity random copolymers. Such organic filler particles may be highly covalently connected to the non-crystalline elastomeric regions of the film, and thus may be effective at reinforcing the film, in particular polyethylene- and polypropylene-based systems. Some filler particles (e.g., titanium dioxide) may also serve as opacifiers (i.e., they improve the opacity of the polymeric film) when incorporated at relatively low levels (e.g., 1 wt. % to 5 wt. %). The filler particles can be coated with a fatty acid (e.g., up to 2 wt. % of stearic acid or a larger chain fatty acid such as behenic acid) to assist dispersion into the polymeric film. The polymeric film may include 30 wt. % to 70 wt. % of the filler particles, for example including 40 wt. % to 60 wt. % filler particles, based on the total weight of the filler particles and the polymeric film.

A method that may improve the breathability of the polymeric film includes the use of discontinuous and/or apertured films. Known methods for creating small apertures either throughout the entire surface area of the film or in discrete regions of the film (e.g., the side panel areas and/or the waistband of an absorbent article) include, for example, mechanical punching or hot-pin aperturing. It is to be understood, however, that any suitable method for creating apertures in a film commonly known to those of ordinary skill in the art is contemplated by at least one embodiment of the invention. The total area formed by the apertures may be between 2% and 20% of the total film surface area, based on trade-offs between breathability, opacity, and load/unload profiles. Pattern selection is largely dictated by the need to minimize stress concentration around the apertures to mitigate the risk of tearing during mechanical activation. Because of the nature of the formulations, the apertures introduced into the film may initially be very small or be in the form of tiny defects which then expand into larger apertures as the polymeric film is stretched. The apertures can be created as part of the film-making process via a vacuum-forming process or a high pressure jet which produces three-dimensional cone-shaped structures around the apertures that help alleviate the risk of tear initiation and propagation during subsequent activation.

Final Processing of the SOC

In embodiments containing the polymeric film, the nonwoven material and the polymeric film may be laminated together with the machine directions of each substantially aligned with the other. The bonding may be accomplished using conventional techniques such as adhesive lamination, extrusion lamination, thermal point bonding, ultrasonic point bonding, adhesive pattern bonding, adhesive spray bonding, and other techniques maintaining the breathability of the film (e.g., those where the bonded areas cover less than 25% of the interface between the polymeric film and nonwoven fibers). The nonwoven material may be partially activated prior to laminate formation. Partial activation of the nonwoven material may reduce the risk of pinhole formation in the film, and thus may facilitate the activation process on the final nonwoven-film laminate.

In another embodiment, a portion of the SOC (e.g., a first spunbond layer and, optionally, a second meltblown layer; a polymeric film) may be pre-stretched in either or both the MD and the CD immediately after being laid and just prior to the addition of more layers to the material. Pre-stretching in the MD can be accomplished by accelerating the web through a set of process rolls. Pre-stretching in the CD can be performed in the same manner as in a tenterframing process, or by using a set of rolls with diverging hills and valleys that force the material outward. Additional SOC layers (i.e., fibrous layers or film layers) may then be added onto the pre-stretched material before being subjected to thermal bonding. The resultant material requires less mechanical activation to exhibit stretch/recovery at any given strain, and it can also minimize the amount of necking during a stretch operation (i.e., size reduction in CD resulting from pulling in the MD). This embodiment may be useful in depositing larger amounts of the additional component per surface area of the nonwoven material in its relaxed state. Pre-stretching can also reduce pinhole formation in the polymeric film in a subsequent activation process.

The outer cover material can be rendered stretchable using a mechanical activation process in both the machine and/or cross machine directions. Such processes typically increase the strain range over which the web exhibits stretch/recovery properties and impart desirable tactile/aesthetic properties to the material (e.g., a cotton-like texture). Mechanical activation processes include ring-rolling, SELFing (differential or profiled), and other means of incrementally stretching webs as known in the art. An example of a suitable mechanical activation process is the ring-rolling process, described in U.S. Pat. No. 5,366,782. Specifically, a ring-rolling apparatus includes opposing rolls having intermeshing teeth that incrementally stretch and thereby plastically deform the material (or a portion thereof) forming the outer cover, thereby rendering the outer cover stretchable in the ring-rolled regions. Activation performed in a single direction (for example the cross direction) yields an outer cover that is uniaxially stretchable. Activation performed in two directions (for example the machine and cross directions or any two other directions maintaining symmetry around the outer cover centerline) yields an outer cover that is biaxially stretchable. In some embodiments, the SOC is activated in at least one region (e.g., a portion of at least one of the front or back waist regions) and remains unactivated in at least one other region, which other region can include a structured elastic-like formed web material.

In some embodiments, the SOC is intentionally activated to differing degrees in different regions (including completely unactivated regions). This manner of processing allows certain regions of the SOC to be elongated to variable extents, thereby permitting the processing of more complex shapes (which in turn reduces the need to trim the SOC into a desired shape). Additionally, a SOC containing unactivated regions can be incorporated into an absorbent article. This permits the consumer to manually stretch the absorbent article (e.g., a diaper), thereby inducing some permanent plastic deformation (i.e., the consumer manually activates the absorbent article) in a manner that provides an improved fit of the absorbent article for the wearer. When the consumer manually activates the absorbent article, absorbent articles manufactured in a single size can comfortably accommodate a wider size range of consumers.

Physical Properties of the SOC

The usefulness of a SOC according to at least one embodiment of the invention relates to a variety of physical properties. The mechanical properties of the SOC relate, for instance, to the ability of the outer cover to survive the high-strain-rate activation process and the ability of an absorbent article incorporating a SOC to conform to a wearer's body in a way that prevents leaks, improves fit, and improves comfort. Underwear-like aesthetic properties such as opacity and texture (e.g., a cotton, ribbed texture) affect consumer appeal for the final absorbent article product. Boys and girls underwear, and also most adult underwear, are typically made of 100% knitted cotton. The ribbed structure of the knitted cotton fabric is at least partially responsible for giving the underwear its desired aesthetics and texture.

Another aspect of underwear-like aesthetics is gloss. A low gloss may give a pleasing matte look (i.e., not plastic like). A gloss value of 7 gloss units or less (as measured according to ASTM D2457-97) has been found desirable. Embossing and/or matte finishing may improve the gloss of the outer cover. Other physical properties such as breathability and liquid permeability may affect comfort of the absorbent article product wearer.

The tensile strain (%) at breaking and % set are relevant mechanical properties. The tensile strain at breaking may be in a range of 200% to 600%, or in a range of 220% to 500%, for example in a range of 250% to 400%. The tensile strain at breaking relates to the ability of the SOC to withstand the activation process and to react to stresses during normal use. The % set of the SOC can be as high as 70% when subjected to a pre-activation Hysteresis Test, and such % set values may allow the SOC simultaneously to be down-gauged (i.e., into a thinner material with a lower basis weight) and/or formed into complex planar or three-dimensional shapes during the activation process. After activation with a strain of 175% (for example with a pair of flat ring-roll plates having a depth of engagement of 2.6 mm and a pitch of 2.5 mm), the first cycle % set of the SOC may be 20% or less or 15% or less, for example 10% or less when subjected a Hysteresis Test having only a 75% strain first loading cycle and a 75% strain second loading cycle. Similarly, prior to any form of activation, the first cycle % set of the SOC may be 20% or less or 15% or less, for example 10% or less when subjected a Hysteresis Test having a 200% strain prestrain loading cycle, a 50% strain first loading cycle, and a 50% strain second loading cycle. The low first cycle % set values (whether post-activation or whether after a prestrain loading cycle that simulates the effect of activation) relate to the ability of the SOC to elastically conform to a wearer's body during use, thereby potentially providing a comfortable and leak-resistant absorbent article. A low-force, recoverable-stretch outer cover may result in an outer cover that is not excessively tight on the baby. In addition, 360 degree stretch in the waist band and leg cuffs may provide the required forces to anchor the product on the body. Further, because the force required to stretch the outer cover to conform to the body of a wearer may be low, only a small amount of elastomer needs to be used; for example, 25 g/m² or even 15 g/m².

A high opacity is a desirable aesthetic property of the SOC, because it provides the consumer with the impression that the SOC will have favorable liquid-retention properties. The opacity of the SOC is preferably at least 65%, more preferably at least 70%, for example at least 75%, in particular when the SOC does not include the polymeric layer.

Even though the absorbent core of an absorbent article typically includes a containment member to limit the escape of liquids, the SOC may be at least partially liquid-impermeable to serve as an additional means for containing waste liquids. Thus, the SOC may be liquid-impermeable to the extent that it has a hydrostatic head (“hydrohead”) pressure up to 80 mbar or 7 mbar to 60 mbar, for example 10 mbar to 40 mbar.

The breathability of a SOC relates to its ability to allow moisture vapor (e.g., water vapor from waste liquid contained in the absorbent core) to permeate the SOC and exit an absorbent article, thereby keeping the wearer's skin dry and free from irritation. The breathability of a SOC is characterized by its moisture vapor transmission rate (“MVTR”). ASTM Method E96-66 provides one suitable method for measuring MVTR. The MVTR of a SOC that includes only nonwoven material and does not include a polymeric film is not particularly limited, and is preferably at least 6,000 g/m² day, with values of at least 9,000 g/m² day being relatively easily attainable. When the SOC includes the polymeric film, which film tends to inhibit vapor transmission, the film often includes filler particles and/or is processed to form apertures so that breathability is improved. For SOCs including the film, the MVTR may be 1,000 g/m² day to 10,000 g/m² day, or 1,000 g/m² day to 6,000 g/m² day, for example 1,200 g/m² day to 4,000 g/m² day.

Test Methods

Hysteresis Test

A commercial tensile tester (e.g., from Instron Engineering Corp. (Canton, Mass.) or SINTECH-MTS Systems Corporation (Eden Prairie, Minn.)) is used for this test. The instrument is interfaced with a computer for controlling the test speed and other test parameters, and for collecting, calculating and reporting the data. The hysteresis is measured under typical laboratory conditions (i.e., room temperature of 20° C. and relative humidity of 50%).

When a SOC is analyzed according to the Hysteresis Test, a 2.54 cm (width)×7.62 cm (length) sample of the SOC material is taken. The length of the SOC sample is taken in the cross machine direction.

The procedure for determining hysteresis is as follows:
1. Select appropriate jaws and a load cell for the test. The jaws must be wide enough to fit the sample (e.g., at least 2.54 cm wide). The load cell is selected so that the tensile response from the sample tested will be between 25% and 75% of the capacity of the load cells or the load range used. A 5-10 kg load cell is typical.
2. Calibrate the tester according to the manufacturer's instructions.
3. Set the gauge length at 25 mm.
4. Place the sample in the flat surface of the jaws such that the longitudinal axis of the sample is substantially parallel to the gauge length direction.
5. Perform the Hysteresis Test with the following steps:
   a. First cycle loading: Pull the sample to 50% strain at a constant cross head speed of 254 mm/min.
   b. First cycle unloading: Hold the sample at 50% strain for 30 seconds and then return the crosshead to its starting position at a constant cross head speed of 254 mm/min. The sample is held in the unstrained state for 1 minute prior to measuring the first cycle % set. If the first cycle % set is not to be measured, the sample can be immediately subjected to the second cycle loading (i.e., nominally 2 seconds after the first cycle unloading).
   c. Second cycle loading: Pull the sample to 50% strain at a constant cross head speed of 254 mm/min.
   d. Second cycle unloading: Hold the sample at 50% strain for 30 seconds and then return crosshead to its starting position at a constant cross head speed of 254 mm/min. The sample is held in the unstrained state for 1 minute prior to measuring the second cycle % set.

A computer data system records the force exerted on the sample during the loading and unloading cycles. From the resulting time-series (or, equivalently, distance-series) data generated, the % set can be calculated. The % set is the relative increase in strain after a given unloading cycle, and this value is approximated by the strain at 0.112 N, measured after the unloading cycle. For example, a sample with an initial length of 10 cm, a prestrain unload length of 15 cm (the prestrain unload length is applicable only to samples subjected to the prestrain cycle, which is described in more detail in example 3), a first unload length of 18 cm, and a second unload length of 20 cm would have a prestrain % set of 50% (i.e., (15−10)/10), a first cycle % set of 20% (i.e., (18−15)/15), and a second cycle % set of 11% (i.e., (20−18)/18). The nominal 0.112 N force is selected to be sufficiently high to remove the slack in a sample that has experienced some permanent plastic deformation in a loading cycle, but low enough to impart, at most, insubstantial stretch to the sample.

The Hysteresis Test can be suitably modified depending on the expected properties of the particular material measured. For instance, the Hysteresis Test can include only some of the loading cycles. Similarly, the Hysteresis Test can include different strains, such as, for example 75% strain, cross head speeds, and/or hold times. However, unless otherwise defined, the term “% set” as recited in the appended claims and examples refers to the first cycle % set as determined by the above loading cycles applied to an unactivated sample.

Modified Hysteresis Test

The Modified Hysteresis Test is identical to the Hysteresis Test described above with the following exceptions: 1) the nominal force applied to remove slack in the sample after the first loading cycle is 0.05 N (instead of 0.112 N) and 2) the slack preload is set at 0 g at the start of this test. The samples were loaded to 50% strain and % set was measured during the second cycle loading curve at a force of 0.05 N.

Tensile to Break Test

A commercial tensile tester (e.g., from Instron Engineering Corp. (Canton, Mass.) or SINTECH-MTS Systems Corporation (Eden Prairie, Minn.)) is used for this test. The instrument is interfaced with a computer for controlling the test speed and other test parameters, and for collecting, calculating and reporting the data. The Peak Elongation is measured under typical laboratory conditions (i.e., room temperature of 20° C. and relative humidity of 50%).

When a SOC is analyzed according to the Tensile to Break test, a 2.54 cm (width)×7.62 cm (length) sample of the SOC material is taken. The length of the SOC sample is taken in the cross machine direction.

Procedure:

1. Select appropriate jaws and a load cell for the test. The jaws must be wide enough to fit the sample (e.g., at least 2.54 cm wide). The load cell is selected so that the tensile response from the sample tested will be between 25% and 75% of the capacity of the load cells or the load range used. A 5-10 kg load cell is typical.
2. Calibrate the tester according to the manufacturer's instructions.
3. Set the gauge length at 25 mm.
4. Place the sample in the flat surface of the jaws such that the longitudinal axis of the sample is substantially parallel to the gauge length direction.
5. Pull the sample at a constant cross head speed of 254 mm/min to 1000% strain or until the sample exhibits a more than nominal loss of mechanical integrity.
   A computer data system records the force exerted on the sample during the test as a function of applied strain. From the resulting data generated, the following quantities are reported:
1. Loads at 15%, 50% and 75% strain (N/cm)
2. Peak elongation (%) and peak load (N/cm)
   Peak elongation is the strain at peak load. Peak load is the maximum load observed during the Tensile to Break test.
   Hydrostatic Head (Hydrohead) Pressure

The property determined by this test is a measure of the liquid barrier property (or liquid impermeability) of a material. Specifically, this test measures the hydrostatic pressure the material will support when a controlled level of water penetration occurs. The hydrohead test is performed according to EDANA 120.2-02 entitled “Repellency: Hydrostatic Head” with the following test parameters. A TexTest Hydrostatic Head Tester FX3000 (available from Textest AG in Switzerland or from Advanced Testing Instruments in Spartanburg, S.C., USA) is used. For this test, pressure is applied to a defined sample portion and gradually increases until water penetrates through the sample. The test is conducted in a laboratory environment at 22±2° C. temperature and 50% relative humidity. The sample is clamped over the top of the column fixture, using an appropriate gasketing material (o-ring style) to prevent side leakage during testing. The area of water contact with the sample is equal to the cross sectional area of the water column, which equals 28 cm². Water inside the column is subjected to a steadily increasing pressure, which pressure increases at a rate of 20 mbar/min. When water penetration appears in three locations on the exterior surface of the sample, the pressure (measured in mbar) at which the third penetration occurs is recorded. If water immediately penetrates the sample (i.e., the sample provided no resistance), a zero reading is recorded. For each material, three specimens are tested and the average result is reported.

Moisture Vapor Transmission Rate Test

This method is applicable to thin films, fibrous materials, and multi-layer laminates of the foregoing. The method is based on ASTM Method E96-66. In the method, a known amount of a desiccant (CaCl₂) is put into a cup-like container. A sample of the outer cover material to be tested (sized to 38 mm x 64 mm, being sufficiently large to cover the opening of the desiccant container) is placed on the top of the container and held securely by a retaining ring and gasket. The assembly is placed in a constant temperature (40° C.) and humidity (75% RH) chamber for 5 hours. The amount of moisture absorbed by the desiccant is determined gravimetrically and used to calculate the moisture vapor transmission rate (MVTR) of the sample. The MVTR is the mass of moisture absorbed divided by the elapsed time (5 hours) and the open surface area at the interface between the container and the sample. The MVTR is expressed in units of g/m²·day. A reference sample, of established permeability, is used as a positive control for each batch of samples. Samples are assayed in triplicate. The reported MVTR is the average of the triplicate analyses, rounded to the nearest 100 g/m²·day. The significance of differences in MVTR values found for different samples can be estimated based on the standard deviation of the triplicate assays for each sample.

Opacity

The opacity value of a material is inversely proportional to the amount of light that can pass through the material. The opacity is determined from two reflectance measurements on a material sample.

To determine the opacity of an outer cover, an appropriately sized sample (based on the measurement opening of the color measurement instrument; a 12 mm diameter for the instrument used herein) is cut from the outer cover and first backed with a black plate. A first color reading is taken with the black-backed sample to determine a first CIE tristimulus value Y₁. The black backing is removed and the sample is then backed with a white plate. A second color reading is taken with the white-backed sample to determine a second CIE tristimulus value Y₂. The opacity is expressed as the ratio of the two readings: Opacity (%)=Y₁/Y₂×100%. The opacity values reported herein were determined with a HUNTERLAB LABSCAN XE (model LSXE, available from Hunter Associates Laboratory, Inc., Reston, Va.). However, other instruments capable of determining CIE tristimulus values are also suitable.

### EXAMPLES

In the following, the properties for each sample prepared for a given example are not necessarily reported for each sample parameter measured. In such case, the omission of a sample from a particular data table indicates that the omitted sample was not evaluated for the properties listed in the data table.

### Example 1

Sample 1A was a spunbond material formed from a layer of elastomeric fibers (“Sₑₗ”; V2120 fiber-grade VISTAMAXX elastomeric polypropylene) having a basis weight of 30 g/m². Sample 1B was a composite nonwoven material formed from a layer of elastic meltblown fibers (“Mₑₗ”; V2120 elastomeric polypropylene) having a basis weight of 4 g/m² in between two layers of elastic spunbond fibers (V2120 elastomeric polypropylene) each having a basis weight of 15 g/m². The spunbond and meltblown fibers had nominal diameters of 20 μm or more and 1 μm, respectively.

Samples 1A and 1B were activated in a hydraulic press using a set of flat plates (pitch of 0.100″ or 2.5 mm), to a depth of engagement of 2.5 mm in either the CD only or in both MD and CD. FIGS. 1 and 2 are the SEMs of Sample 1B prior to and after activation, respectively. The changes in sample dimensions produced during mechanical activation were subsequently subjected to a Hysteresis Test omitting the prestrain loading cycle to determine the post-activation, first cycle % set, and the results are summarized in Table 1

TABLE 1

| | | | | % Set (CD) After |
|---|---|---|---|---|
| | | Basis | % Set (CD) After | Activation in |
| Sample | Material | Weight | Activation in CD | MD/CD |
| 1A | Sₑₗ | 30 g/m² | 21.0% | 21.3% |
| 1B | SₑₗMₑₗSₑₗ | 34 g/m² | 11.0% | 11.9% |

The results in Table 1 illustrate the ability of the interlayer meltblown fibers to increase the ability of the nonwoven to undergo recovery of the SOC by substantially reducing the % set produced during activation. They suggest that the meltblown layer helps maintain the mechanical integrity of the nonwoven material during mechanical activation. In both cases, the softness of the nonwoven material is improved after activation.

### Example 2

Sample 2A was a spunbond material formed from two superimposed layers of elastomeric fibers (V2120 fiber-grade VISTAMAXX elastomeric polypropylene) each having a basis weight of 30 g/m². Sample 2B was a thermally bonded composite nonwoven material formed from a layer of elastic nanofibers (“Nₑₗ”; V2120 elastomeric polypropylene) having a basis weight of 5 g/m² in between two layers of elastic spunbond fibers (V2120 elastomeric polypropylene) each having basis weight of 30 g/m². The spunbond and meltblown fibers had nominal diameters of 20 μm or more and less than 1 μm, respectively.

Samples 2A and 2B were analyzed according to the opacity test. FIG. 3 is the SEM of Sample 2B prior to mechanical activation. The results are summarized in Table 2.

TABLE 2

| Sample | Material | Basis Weight | Opacity (%) |
|---|---|---|---|
| 2A | Sₑₗ | 60 g/m² | 43% |
| 2B | SₑₗNₑₗSₑₗ | 65 g/m² | 52% |

The results in Table 2 illustrate the ability of the interlayer nanofibers to improve the aesthetic properties of the SOC by substantially increasing the opacity of the nonwoven material. Based on this data, a projected total of 10 g/m² to 20 g/m², for example 15 g/m² of meltblown fibers would suffice to reach an opacity of at least 65% for the nonwoven material, prior to activation, in the relaxed state.

### Example 3

The samples of Example 3 illustrate the tensile properties of nonwoven plastoelastic materials formed from a mixture of elastomeric fibers (V2120 fiber-grade VISTAMAXX elastomeric polypropylene) and plastic fibers (polyolefin-based). Table 3A lists the various samples tested, the approximate relative amounts of elastomeric fibers and plastic fibers in each sample, and the nominal basis weights of the mixed fiber sample.

TABLE 3A

| | | Elastomeric | |
|---|---|---|---|
| Sample | Target Basis Weight | Component | Plastic Component |
| 3A | 25 g/m² | 100 wt. % | 0 wt. % |
| 3B | 25 g/m² | 50 wt. % | 50 wt. % |
| 3C | 35 g/m² | 50 wt. % | 50 wt. % |
| 3D | 45 g/m² | 50 wt. % | 50 wt. % |
| 3E | 25 g/m² | 58 wt. % | 42 wt. % |
| 3F | 35 g/m² | 58 wt. % | 42 wt. % |
| 3G | 45 g/m² | 58 wt. % | 42 wt. % |

The tensile properties of Samples 3B-3G were tested after activation in both the CD and MD using a set of flat plates placed in a hydraulic press. Activation was performed at intermediate strain rate values and a depth of engagement of 2.5 mm. Table 3B summarizes results in terms of the sample tested, its actual basis weight, and the direction in which the tensile property was determined. The tensile properties were determined using standard EDANA methods and an MTS ALLIANCE RT 1/2 tensile testing apparatus (available from MTS Systems Corp., Eden Prairie, Minn.) equipped with pneumatic grips operating at 254 mm/min for a gage length of 25 mm and a sample width of 25 mm.

TABLE 3B

| | Actual | | Peak Load | Peak Stress | Strain at |
|---|---|---|---|---|---|
| Sample | Basis Weight | Direction | (N/cm) | (MPa) | Break (%) |
| 3B | 25 g/m² | CD | 2.47 | 9.07 | ˜300-400 |
| 3C | 36 g/m² | CD | 4.21 | 10.3 | 326 |
| 3D | 49 g/m² | CD | 5.43 | 10.0 | ˜300-400 |
| 3E | 26 g/m² | CD | 2.01 | 7.00 | ˜350-400 |
| 3E | 25 g/m² | MD | 5.71 | 21.1 | 235 |
| 3F | 36 g/m² | CD | 3.60 | 8.84 | 329 |
| 3G | 46 g/m² | CD | 4.99 | 9.60 | 285 |

Samples 3A and 3E were also subjected a Hysteresis Test, the results of which are shown in Table 3C. The “% set” value is the first cycle % set. The samples were subjected to the Hysteresis Test as described in the Test Methods section, with the exception that the pre-activated samples were not prestrained during the test. The “maximum load” value represents either the force at 200% strain for the unactivated sample during the prestrain cycle or the force at 75% strain for the activated samples during the first loading cycle. The activated samples were tested after activation in both the CD and MD in a benchtop hydraulic press having a depth of engagement of 2.5 mm.

TABLE 3C

| | | | | | 1^{st} Strain | | 2^{nd} Strain | |
|---|---|---|---|---|---|---|---|---|
| | | Actual | | | Cycle | | Cycle | |
| | | Basis | % | Maximum | 50% | 75% | 20% | 75% |
| Sample | Act. | Weight | Set | Load | Load | Relax. | Load | Relax. |
| 3A | N | 25 g/m² | 33.4 | 3.09 N | 0.37 N | 46.5% | 0.04 N | 36.2% |
| 3A | Y | 18 g/m² | 17.2 | 0.64 N | 0.26 N | 50.6% | 0.03 N | 35.5% |
| 3E | Y | 24 g/m² | 25.7 | 0.64 N | 0.25 N | 47.9% | 0.01 N | 33.7% |

Samples 3E-3G were also subjected to a high strain rate activation test, using a High-Speed Research Press (“HSRP”). During the test, the force applied to a nonwoven material sample was measured while the material was elongated up to a strain of 1000% at strain rates up to 1000 s⁻¹ using two flat ring-roll plates having a depth of engagement of 8.2 mm and a pitch of 1.5 mm. The samples were essentially completely shredded at the end of the test. The resulting data (i.e., applied force as a function of strain at a fixed strain rate) were analyzed to identify the strain at which the applied force was at a maximum. When the normalized applied force (i.e., applied force per unit weight of the nonwoven sample) is at a maximum, the nonwoven material loses its ability to withstand additional loading without an increased likelihood of material destruction. The strain at the maximum applied force represents the ability of the nonwoven material to withstand the mechanical activation process having approximately the same degree of strain. Table 3D summarizes the results of these tests.

TABLE 3D

| | | | Maximum | |
|---|---|---|---|---|
| | | Strain | Applied | Strain at |
| Sample | Strain Rate | Direction | Force | Max. Force |
| 3E | 1000 s⁻¹ | CD | 17 kN/g | 200% |
| 3F | 1000 s⁻¹ | CD | 18 kN/g | 200% |
| 3G | 1000 s⁻¹ | CD | 19 kN/g | 190% |
| 3E | 500 s⁻¹ | MD | 35 kN/g | 180% |
| 3E | 500 s⁻¹ | CD | 15 kN/g | 280% |

The results in Table 3D suggest that the plastoelastic materials of the present disclosure are capable of withstanding a mechanical activation process at strain levels up to 200%, for example up to 300%, while incurring only minimal damage, even at very high strain rate conditions. This is in contrast to typical commercial extensible nonwoven materials that can only withstand strains up to 150% when subjected to comparable strain rates.

The activation process also improves the softness and feel of the plastoelastic nonwoven material. This effect is largely related to the increase in web loft/thickness created during the activation process. FIGS. 6-9 illustrate this effect for the nonwoven plastoelastic materials of Example 3. FIGS. 6 and 7 are SEMs of a bonded plastoelastic nonwoven material prior to activation (top and side views, respectively). FIGS. 8 and 9 are SEMs of the same nonwoven material after activation (top and side views, respectively), and they illustrate the increased thickness of the material.

### Example 4

The samples of Example 4 illustrate the tensile properties of composite nonwoven plastoelastic materials formed from a layer of plastoelastic bi-component spunbond fibers and a layer of elastic spunbond fibers. V2120 fiber-grade VISTAMAXX elastomeric polypropylene was used as the elastic component of the bi-component fibers and for the elastic fibers themselves. For samples 4A-4D, the plastic component of the bi-component fibers was a mixture of PH-835 Ziegler-based polypropylene (50 wt. %; available from Basell Polyolefins, Elkton, Md.) and HH-441 high melt flow rate polypropylene (50 wt. %; melt flow rate=400 g/10 minutes; available from Himont Co., Wilmington, Del.). For samples 4E-4G, the plastic component of the bi-component fibers was a Basell Moplen 1669 random polypropylene copolymer with a small amount of polyethylene (also available from Basell Polyolefins). The bi-component fibers had an elastomeric core and a plastic sheath, and the weight fraction of each component is given in Table 4. The elastic fibers also contained 3.5 wt. % of an anti-blocking agent to improve their spinning performance. Each of the two spunbond layers represents half of the total basis weight of the nonwoven material (i.e., the value listed in the second column of Table 4). The two spunbond layers were thermally bonded using two heated rolls, with the first at 84° C., and the second at 70° C.

Table 4 summarizes the tensile properties of the spunbond-spunbond composites tested in an unactivated state. The properties were determined with standard EDANA methods (EDANA method 40.3-90 for the basis weight and EDANA method 20.2-89 for the tensile properties).

Table 4 also summarizes properties of the composites as measured by a hysteresis test. The Hysteresis Test described in the “Test Methods” section above was modified in the following aspects: (1) sample size (5 cm wide×15 cm long), (2) crosshead speed (500 mm/min), (3) prestrain loading/unloading (omitted), and (4) first and second cycle loading/unloading 5 (100% maximum strain, held for 1 second at maximum strain, held for 30 seconds after unloading). For each cycle, Table 4 provides the force at 100% strain (normalized by the sample width) and the % set after unloading. For the first cycle, the % set is the strain after the first cycle unloading. For the second cycle, the % set is the relative increase in strain between the unloaded states of the first and second cycles. For example, a sample with an initial length of 10 cm, a first unload length of 15 cm, and a second unload length of 18 cm would have a first cycle % set of 50% and a second cycle % set of 20%.

TABLE 4

| | | Core/ | Tensile | | | | Load at | | | |
|---|---|---|---|---|---|---|---|---|---|---|
| | | Sheath | Stress | | | | 100% Strain | | | |
| | Basis | Weight | (N/50 | | Elongation | | (N/50 mm) | | % Set | |
| | Wt. | Ratio | mm) | | (%) | | 1^{st} | 2^{nd} | 1^{st} | 2^{nd} |
| Sample | (g/m²) | (%/%) | CD | MD | CD | MD | Cycle | Cycle | Cycle | Cycle |
| 4A | 37.5 | 80/20 | 11.9 | 17.9 | 106 | 101 | 11.4 | 9.58 | 70 | 17 |
| 4B | 38.8 | 90/10 | 8.50 | 12.8 | 152 | 155 | 7.68 | 6.76 | 59 | 19 |
| 4C | 58.7 | 80/20 | 20.2 | 29.2 | 133 | 139 | 18.7 | 16.4 | 68 | 20 |
| 4D | 60.7 | 90/10 | 18.7 | 24.2 | 144 | 133 | 14.4 | 12.7 | 57 | 21 |
| 4E | 44.8 | 90/10 | 8.00 | 11.0 | 145 | 133 | 6.70 | 5.80 | 45 | 8 |
| 4F | 66.7 | 90/10 | 14.6 | 18.7 | 158 | 146 | 12.9 | 11.0 | 52 | 16 |
| 4G | 59.7 | 80/20 | 18.0 | 24.8 | 102 | 100 | 18.1 | 15.7 | 61 | 17 |

The results in Table 4 indicate that a mechanically activated SOC formed from the plastoelastic materials of the present disclosure has favorable stretch properties, and would be able to exhibit % set values less than 20%, and as low as less than 10%.

### Example 5

The samples of Example 5 illustrate the tensile properties of plastoelastic film materials formed with an elastomeric component (V1100 film-grade VISTAMAXX elastomeric polypropylene), plastic components (polyolefin-based), and an optional opacifier. The various plastic components are summarized in Table 5A and include linear low density polyethylene (LL6201), low molecular weight polyethylene waxes (A-C 617, A-C 735, and PARVAN 1580), and a low molecular weight polypropylene wax (LICOWAX PP230). The unactivated samples were tested to determine their tensile properties and then subjected to a Hysteresis Test with the following modification: the test included only a prestrain and a first cycle loading (with a maximum strain of 50% and a 30 second hold time. The results of this test are provided in Tables 5B and 5C. It should be noted that the Sample designations represent a sample prepared according to the formulation shown in the table. The sample is then subjected to a particular test. As a result, the physical parameters of the samples, such as basis weight, may vary even though the sample designation is the same. For example, Sample 5E shown in Table 5B lists a different basis weight than Sample 5E in Table 5C.

TABLE 5A

| | | | | | | | TiO₂ |
|---|---|---|---|---|---|---|---|
| | V1100 | LL6201 | AC 735 | AC 617 | P. 1580 | PP 230 | (wt. |
| Sample | (wt. %) | (wt. %) | (wt. %) | (wt. %) | (wt. %) | (wt. %) | %) |
| 5A | 60 | 10 | 10 | | | 20 | |
| 5B | 60 | 10 | | 10 | | 20 | |
| 5C | 60 | 10 | | | 10 | 20 | |
| 5D | 58.8 | 9.8 | | | 9.8 | 19.6 | 2.0 |
| 5E | 85 | 15 | | | | | |

TABLE 5B

| | Basis | | Peak Load | Peak Stress | Strain at |
|---|---|---|---|---|---|
| Sample | Weight | Direction | (N/cm) | (MPa) | Break (%) |
| 5A | 16 g/m² | CD | 6.8 | 15 | 741 |
| 5B | 24 g/m² | CD | 10.5 | 14 | 636 |
| 5C | 19 g/m² | CD | 8.0 | 15 | 755 |
| 5E | 29 g/m² | CD | 20.7 | 23 | 848 |

TABLE 5C

| | | | | | 1^{st} Strain Cycle | | |
|---|---|---|---|---|---|---|---|
| | Film | | | Prestrain | | | |
| | Thick- | Basis | | 200% | 50% | 50% | 30% |
| Sample | ness | Weight | % Set | Load | Load | Relax. | Unload |
| 5A | 13 μm | 16 g/m² | 33.7 | 1.36 N | 0.6 N | 31.5% | 0.15 N |
| 5B | 22 μm | 24 g/m² | 27.3 | 2.07 N | 0.9 N | 30.7% | 0.25 N |
| 5C | 20 μm | 20 g/m² | 41.8 | 2.03 N | 0.9 N | 33.9% | 0.20 N |
| 5D | 25 μm | 24 g/m² | 32.3 | 2.50 N | 1.1 N | 32.7% | 0.23 N |
| 5E | 13 μm | 14 g/m² | 32.0 | 1.50 N | 0.5 N | 76.1% | 0.05 N |

The results in Table 5A-5C illustrate that the plastoelastic film formulations of the present disclosure have favorable mechanical properties that make them suitable for inclusion into a SOC.

### Example 6

The samples of Example 6 illustrate the tensile properties of an elastic film formed with elastomeric components, anti-blocking agents, and an opacifier (titanium dioxide). The various components are summarized in Table 6A and include elastomeric polypropylene (V 1100 film-grade VISTAMAXX), styrenic block copolymers (VECTOR V4211 and PS3190 (available from Nova Chemicals, Pittsburgh, Pa.)), a soft polypropylene-based thermoplastic elastomer reactor blend (ADFLEX 7353, available from Basell Polyolefins, Elkton, Md.), and anti-blocking agents (CRODAMIDE and INCROSLIP, both available from Croda, Inc., Edison, N.J.). The unactivated samples were tested to determine their tensile properties and then subjected to a Hysteresis Test modified as described in example 5 (i.e., including only a prestrain and a first cycle loading (with a maximum strain of 50% and a 30 second hold time)), the results of which are provided in Tables 6B and 6C. It should be noted that the Sample designations represent a sample prepared according to the formulation shown in the table. The sample is then subjected to a particular test. As a result, the physical parameters of the samples, such as basis weight, may vary even though the sample designation is the same. For example, Sample 6B shown in Table 6B lists a different basis weight than Sample 6B in Table 6C.

TABLE 6A

| | V1100 | V4211 | PS3190 | Adflex | Crodamide | Incroslip B | TiO₂ |
|---|---|---|---|---|---|---|---|
| Sample | (wt. %) | (wt. %) | (wt. %) | (wt. %) | (wt. %) | (wt. %) | (wt. %) |
| 6A | 41.7 | 37.0 | 6.5 | | 5.55 | 5.55 | 3.7 |
| 6B | 75.6 | | | 8.4 | 5.5 | 6.8 | 3.7 |
| 6C | 85.7 | | | | 4.0 | 6.7 | 3.6 |

TABLE 6B

| | Basis | | Peak Load | Peak Stress | Strain at |
|---|---|---|---|---|---|
| Sample | Weight | Direction | (N/cm) | (MPa) | Break (%) |
| 6A | 31 g/m² | CD | 16.5 | 21 | 731 |
| 6B | 25 g/m² | CD | 11.0 | 15 | 623 |

TABLE 6C

| | | | | | 1^{st} Strain Cycle | | |
|---|---|---|---|---|---|---|---|
| | Film | | | Prestrain | | | |
| Sam- | Thick- | Basis | | 200% | 50% | 50% | 30% |
| ple | ness | Weight | % Set | Load | Load | Relax. | Unload |
| 6A | 25 μm | 31 g/m² | 11.6 | 2.30 N | 1.17 N | 21.6% | 0.51 N |
| 6B | 20 μm | 21 g/m² | 14.8 | 1.70 N | 0.90 N | 21.1% | 0.39 N |
| 6C | 20 μm | 21 g/m² | 19.2 | 1.86 N | 0.90 N | 23.1% | 0.35 N |

The results in Tables 6A-6C illustrate that the elastic film formulations of the present disclosure have favorable mechanical properties that make them suitable for inclusion into a SOC when combined with a nonwoven material into a laminate structure.

### Example 7

The samples of Example 7 illustrate the effect of including a plasticizer on the tensile properties of an elastic film. The various components are summarized in Table 7A. The plasticizer used was mineral oil, and the mineral oil was added to the formulation by heating the V1100 elastomeric polypropylene at 50° C. while in contact with the oil. The unactivated samples were then subjected to a Hysteresis Test (modified as described in examples 5 and 6), the results of which are provided in Table 7B.

TABLE 7A

| | V1100 | Min. Oil | Crodamide | Incroslip B | TiO₂ |
|---|---|---|---|---|---|
| Sample | (wt. %) | (wt. %) | (wt. %) | (wt. %) | (wt. %) |
| 6A | 80 | | 6 | 6 | 8 |
| 6C | 60 | 20 | 6 | 6 | 8 |

TABLE 7B

| | | | | 1^{st} Strain Cycle | | | |
|---|---|---|---|---|---|---|---|
| | Film | | | Prestrain | | | |
| Sam- | Thick- | Basis | | 200% | 50% | 50% | 30% |
| ple | ness | Weight | % Set | Load | Load | Relax. | Unload |
| 7A | 20 μm | 21 g/m² | 19.2 | 1.86 N | 0.9 N | 23.1% | 0.35 N |
| 7B | 15 μm | 14 g/m² | 17.9 | 0.48 N | 0.2 N | 17.8% | 0.11 N |

The results in Tables 7A-7B illustrate that the inclusion of a plasticizer into the film formulations of the present disclosure can substantially reduce the loading/unloading forces while retaining favorable % set values.

### Example 8

The samples of Example 8 illustrate the effect of including filler particles on the breathability and the tensile properties of a plastoelastic film formed with an elastomeric component (V1100 film-grade VISTAMAXX elastomeric polypropylene and, optionally, VECTOR V4211 styrenic block copolymer), a plastic component (LL6201 linear low density polyethylene), calcium carbonate filler particles, and titanium dioxide opacifying particles. The samples were tested after activation in the CD only at strain rates of 500 s⁻¹ and a depth of engagement of 4.4 mm for a pitch of 3.8 mm (0.150″). The formulations and resulting properties are show in Tables 8A and 8B. The samples listed in Table 8B were subjected to a Hysteresis Test (modified as described in examples 5 and 6).

TABLE 8A

| | | | | | | Film | |
|---|---|---|---|---|---|---|---|
| | | | | | | Thick- | MVTR |
| Sam- | V1100 | V4211 | LL6201 | CaCO₃ | TiO₂ | ness | (g/m² · |
| ple | (wt. %) | (wt. %) | (wt. %) | (wt. %) | (wt. %) | (μm) | d) |
| 8A | 30 | | 20 | 48 | 2 | 30 | 1727 |
| 8B | 32 | | 16 | 50 | 2 | 30 | 2064 |
| 8C | 33 | | 13 | 52 | 2 | 46 | 1746 |
| 8D | 34 | | 10 | 54 | 2 | 33 | 1908 |
| 8E | 35 | | 7 | 56 | 2 | 30 | 1056 |
| 8F | 38 | | | 60 | 2 | 48 | 206 |
| 8G | 37 | | 10 | 51 | 2 | 25 | 348 |
| 8H | 44 | | 10 | 44 | 2 | 25 | 197 |
| 8I | 42 | | 10 | 46 | 2 | 38 | 209 |
| 8J | 28 | 6 | 10 | 54 | 2 | 25 | 2989 |

TABLE 8B

| | | | 1^{st} Strain Cycle | | | |
|---|---|---|---|---|---|---|
| | | | Prestrain | | | |
| | Basis | | 200% | 50% | 50% | 30% |
| Sample | Weight | % Set | Load | Load | Relax. | Unload |
| 8A | 43 g/m² | 55.3 | 3.31 N | 2.0 N | 33.9% | 0.26 N |
| 8B | 41 g/m² | 51.1 | 3.22 N | 1.8 N | 33.4% | 0.26 N |
| 8C | 59 g/m² | 65.5 | 4.02 N | 2.6 N | 35.9% | 0.36 N |
| 8D | 48 g/m² | 36.3 | 2.93 N | 1.3 N | 31.2% | 0.29 N |
| 8E | 42 g/m² | 30.0 | 2.30 N | 1.0 N | 28.9% | 0.27 N |
| 8F | 68 g/m² | 26.1 | 3.34 N | 1.4 N | 28.0% | 0.43 N |

The results in Tables 8A-8B illustrate that the inclusion of filler particles into the film formulations of the present disclosure can substantially increase the breathability of the film while retaining favorable mechanical properties.

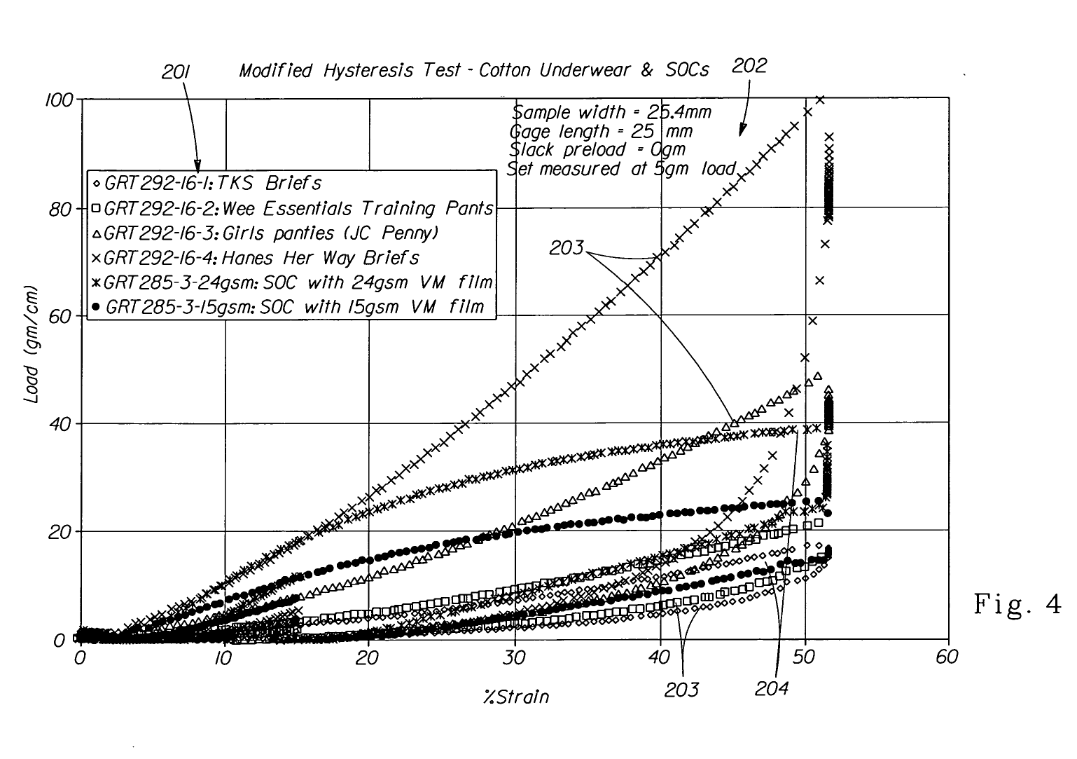

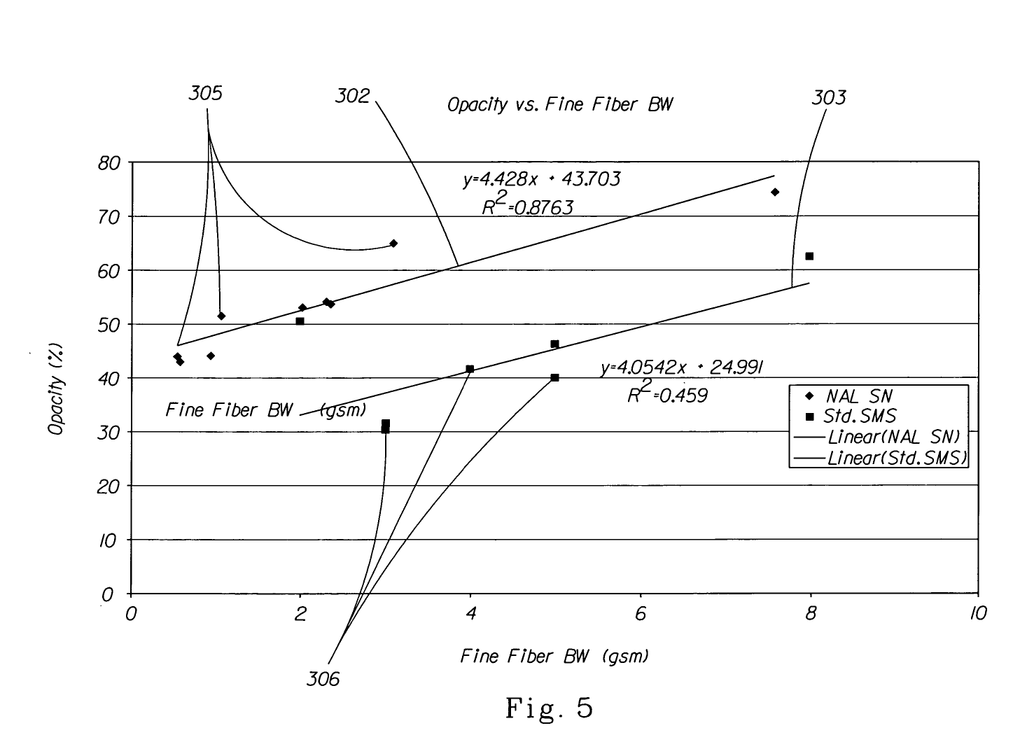

Table 9 and FIG. 4 show comparative data for 6 samples 201. The data graphs 202 of the results can be seen in FIG. 4. The samples 201 included four commercial brands of underwear 203 and two stretchable outer covers 204 according to at least one embodiment of the invention. The samples 201 were measured according to the Modified Hysteresis Test described in the Test Methods section. The measurements on the underwear samples 203 were made in the lateral direction (i.e., the direction substantially parallel to the waistband of the underwear). Commercial underwear 203 typically have more stretch in the lateral direction than the longitudinal direction, but still exhibit suitable low-force, recoverable-stretch properties in the longitudinal direction.

TABLE 9

| | | First Cycle Load | | | |
|---|---|---|---|---|---|
| | | at given | | | |
| | | strain (gm/cm) | | | % set |
| ID | Description | 15% | 25% | 50% | .05 N |
| Target | | <20 | | <40 | <20 |
| GRT292- | TKS Basics Toddler Boys | 3.0 | 5.7 | 17.5 | 14.9 |
| 16-1 | Brief 2T/3T | | | | |
| GRT292- | WEE ESSENTIALS Padded | 3.4 | 7.1 | 21.1 | 14.8 |
| 16-2 | Training Pants, 3T | | | | |
| | (Distributed by JC PENNEY) | | | | |
| GRT292- | JC PENNEY White Panties | 8.1 | 16.5 | 47.6 | 11.3 |
| 16-3 | Girl, 2T/3T, # 344 1110800305 | | | | |
| GRT292- | HANES HER WAY | 18.1 | 36.6 | 97.8 | 10.7 |
| 16-4 | CLASSICS Brief Size 4 | | | | |
| | (UPC: 75338 30388) | | | | |
| GRT285- | 24 g/m² solid VISTAMAXX | 18.6 | 28.3 | 39.2 | 7.9 |
| 3-24 g/m² | 1100 film + H2031 adhesive + 2 | | | | |
| | layers of 25 g/m² DAPP | | | | |
| | NW; Activation in the | | | | |
| | hydraulic press (P = 0.100″, | | | | |
| | DOE = 0.158″) | | | | |
| GRT285- | 15 g/m² solid VISTAMAXX | 9.8 | 17.1 | 25.4 | 7.8 |
| 3-15 g/m² | 1100 film + H2031 adhesive + 2 | | | | |
| | layers of 25 g/m² DAPP | | | | |
| | NW; Activation in the | | | | |
| | hydraulic press (P = 0.100″, | | | | |
| | DOE = 0.158″) | | | | |

Table 10 and FIG. 9 show comparative opacity data for various basis weight nonwoven substrates. FIG. 9 shows a nanofiber trendline 302 and a standard meltblown fiber trendline 303. The nanofiber trendline 302 was produced from the nanofiber datapoints 305 corresponding to the nanofiber substrates labeled as samples 1-9 in Table 10. Samples 1-10 in Table 10 correspond to an unbonded spundbond-nanofiber-spunbond substrate. The basis weights for each individual layers is listed in the ID column. The basis weights were measured in gram per square meter (“gsm”). The Total Basis weight corresponds to the sum of the individual layer basis weights. The standard meltblown fiber trendline 303 was produced from the standard meltblown datapoints 306 corresponding to the standard meltblown substrates labeled as sample 11-17 in Table 10. The standard meltblown fiber substrates are commercially available substrates. The basis weight of each layer is listed in the ID column. As can be seen from the data a nonwoven substrate comprising nanofibers may provide improved opacity over a standard nonwoven substrate for a given basis weight.

TABLE 10

| | | Fine | Total | |
|---|---|---|---|---|
| | | Fiber | Basis | |
| Sample | | BW | weight | |
| # | ID | (gsm) | (gsm) | Opacity |
| 1 | SN + S 13.5/2.36/13.5 UNBONDED | 2.36 | 29.4 | 53.9 |
| 2 | SN + S 13.5/2.03/13.5 UNBONDED | 2.03 | 29 | 53.2 |
| 3 | SN + S 13.5/7.6/13.5 UNBONDED | 7.6 | 34.6 | 74.4 |
| 4 | SN + S 13.5/0.55/13.5 UNBONDED | 0.55 | 27.6 | 44.2 |
| 5 | SN + S 13.5/1.07/13.5 UNBONDED | 1.07 | 28.1 | 51.7 |
| 6 | SN + S 13.5/3.1/13.5 UNBONDED | 3.1 | 30.1 | 65.1 |
| 7 | SN + S 12/4/03 2:35 13.5/0.94/13.5 | 0.94 | 27.9 | 44.2 |
| | UNBONDED | | | |
| 8 | SN + S 12/4/03 2:44 13.5/2.31/13.5 | 2.31 | 29.3 | 54.3 |
| | UNBONDED | | | |
| 9 | SN + S 12/4/03 2:26 13.5/0.58/13.5 | 0.58 | 27.6 | 43.2 |
| | UNBONDED | | | |
| 10 | FIBERTEX 22GSM 10/1/1/10 | 2 | 22 | 50.6 |
| | H1502220 W/TIO2 | | | |
| 11 | FQN 7/3/7 SMS | 3 | 17 | 30.4 |
| 12 | FQN HIGH OPACITY 7.5/5/7.5 W/ | 5 | 20 | 46..3 |
| | TIO2 | | | |
| 13 | FQN C123 SMS 11/8/11 W/TIO2 | 8 | 30 | 62.4 |
| 14 | FQN SBC SMS 6/5/6 MB = 2.5MIC | 5 | 17 | 40.2 |
| 15 | 7/3/7 SMS FIBERTEX | 3 | 17 | 31.6 |
| | ELITE(1.5 MB, 12 MB) | | | |
| 16 | 30 (13/4/13) SMS FQN | 4 | 30 | 41.6 |
| 17 | 7/3/7 SMS BBA TORONTO | 3 | 17 | 30.4 |

The dimensions and values disclosed herein are not to be understood as being strictly limited to the exact numerical values recited. Instead, unless otherwise specified, each such dimension is intended to mean both the recited value and a fimctionally equivalent range surrounding that value. For example, a dimension disclosed as “40 mm” is intended to mean “40 mm.”

All documents cited in the Detailed Description of the Invention are, in relevant part, incorporated herein by reference; the citation of any document is not to be construed as an admission that it is prior art with respect to at least one embodiment of the invention. To the extent that any meaning or definition of a term in this written document conflicts with any meaning or definition of the term in a document incorporated by reference, the meaning or definition assigned to the term in this written document shall govern.

While particular embodiments of the invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stretchable outer cover for an absorbent article comprising:
a multi-layered elastomeric film layer including
a. at least one skin layer, the skin layer being at least one of elastomeric and plastoelastic; and
b. at least one elastomeric core layer, the elastomeric core layer including a first elastomeric polypropylene,
wherein the skin layer is less tacky than the core layer.

2. The stretchable outer cover of claim 1, wherein at least one of the skin layer and the core layer include at least one antiblock additive and the weight % of the antiblock additive in the skin layer based on the total weight of the skin layer is higher than the weight % of antiblock additive in the core layer based on the total weight of the core layer.

3. The stretchable outer cover of claim 1, wherein the skin layer is elastomeric and includes a second elastomeric polypropylene, the second elastomeric polypropylene having a higher degree of crystallinity than the first elastomeric polypropylene.

4. The stretchable outer cover of claim 1, wherein the skin layer is elastomeric and includes a second elastomeric polypropylene, the second elastomeric polypropylene having at least one higher melting temperature than the first elastomeric polypropylene.

5. The stretchable outer cover of claim 1, further comprising at least one nonwoven layer.

6. The stretchable outer cover of claim 1, wherein the multilayered elastomeric film includes a styrenic block copolymer.

7. An underwear-like, low-force, recoverable stretch outer cover for an absorbent article, the outer cover comprising:
a. an elastomeric film; and
b. at least one nonwoven
wherein the outer cover has a first cycle load at 15% strain of less than 40 g/cm and a % set of less than 20% in at least the cross direction as measured according to the Modified Hysteresis Test.

8. The stretchable outer cover of claim 7, wherein the basis weight of the elastomeric film is less than 30 g/m².

9. The stretchable outer cover of claim 7, wherein the first cycle load at 15% strain is less than 20 g/cm.

10. The stretchable outer cover of claim 7, wherein the elastomeric film comprises an elastomeric polypropylene composition.

11. The stretchable outer cover of claim 10, wherein the elastomeric polypropylene composition comprises an elastomeric random copolymer including propylene with a low level of comonomer incorporated into the backbone.

12. The stretchable outer cover of claim 11, wherein the comonomer comprises an α-olefin selected from the group consisting of ethylenes, propylenes, and butenes.

13. The stretchable outer cover of claim 7, wherein the outer cover has a gloss value of less than 7 units.

14. The stretchable outer cover of claim 7, wherein the outer cover has an MVTR greater than 1000 gm/m²/day.

15. The stretchable outer cover of claim 7, wherein the elastomeric film is apertured.

16. The stretchable outer cover of claim 7, wherein the elastomeric film is microporous.

17. The stretchable outer cover of claim 7, wherein the outer cover has an opacity of greater than 65% when measured according to the Opacity Test.

18. The stretchable outer cover of claim 7, wherein the outer cover is elastic.

19. The stretchable outer cover of claim 7, wherein the outer cover is activated at least in one direction

20. The stretchable outer cover of claim 7, wherein the outer cover has a ribbed texture.

21. The stretchable outer cover of claim 7, wherein the elastomeric film is joined to the nonwoven using an adhesive to form a laminate.

22. The stretchable outer cover of claim 21, wherein the adhesive comprises an elastomeric component selected from the group consisting of elastomeric polyolefins and styrenic block copolymers.

23. The stretchable outer cover of claim 7, wherein the first cycle load at 50% strain is less than 75 g/cm.

24-36. (canceled)

37. A process for making a stretchable outer cover for an absorbent article, the process comprising:
a. providing at least one non-elastic nonwoven substrate;
b. joining an elastomeric film comprising an elastomeric polypropylene to the nonwoven to form a laminate;
c. aperturing the laminate using mechanical aperturing or hot pins; and
d. activating at least a portion of the laminate in at least the cross direction.

38. The process of claim 37, wherein the laminate further comprises a third layer, the third layer including a nonwoven and being configured such that the second layer is disposed between the first layer and the third layer.

39. The process of claim 37, wherein the elastomeric film is extruded onto the nonwoven

40. The process of claim 37, wherein the elastomeric film is adhesively joined to the nonwoven.

41. The process of claim 37, wherein the elastomeric film is prestretched at least in one direction prior to being joined to the nonwoven.

42. The process according to claim 37, wherein at least a portion of the laminate is activated in the machine direction.

43. The process according to claim 37, wherein the basis weight of the elastomeric film is less than 30 g/m².

44. The process according to claim 37, wherein the elastomeric film and the nonwoven are bonded to each other by way of at least one of adhesive bonding, thermal point bonding, and ultrasonic point bonding.

45. The process according to claim 37, wherein the elastomeric film comprises at least one elastomeric core layer and at least one skin layer, the skin layer being either elastomeric or plastoelastic.

46. A wearable absorbent article for receiving and storing bodily exudates comprising:
a. a liquid permeable nonwoven topsheet comprising a plurality of fibers;
b. an underwear-like, stretchable, multi-layered outer cover including comprising:
i. at least one skin layer, the skin layer being at least one of elastomeric and plastoelastic;
ii. at least one elastomeric core layer, the elastomeric core layer including a first elastomeric polypropylene, wherein the skin layer is less tacky than the core layer.
iii. at least one nonwoven;
iv. a first cycle load at 15% strain of less than 40 g/cm and % set of less than 20% in at least the cross direction as measured according to the Modified Hysteresis Test; and
c. an absorbent core disposed between the topsheet and the outer cover.

47. The disposable absorbent article of claim 46, wherein the outer cover has an opacity of greater than 65% when measured according to the Opacity Test.

48. The disposable absorbent article of claim 46, further comprising at least one nanofiber layer.

49. The disposable absorbent article of claim 48, wherein the nanofiber layer comprises meltblown elastomeric fibers.

50. The disposable absorbent article of claim 49, wherein the elastomeric fibers comprise an elastomeric polypropylene.

51. The disposable absorbent article of claim 50, wherein the elastomeric polypropylene comprises an elastomeric random copolymer including propylene with a low level of comonomer incorporated into the backbone.

52. The disposable absorbent article of claim 46, wherein the comonomer comprises an α-olefin selected from the group consisting of ethylenes, propylenes, and butenes.

53. The disposable absorbent article of claim 46; wherein the outer cover has a ribbed texture.

54. The disposable absorbent article of claim 46, wherein the outer cover has a gloss value of less than 7 units.

55. The disposable absorbent article of claim 46, wherein the fibers comprise a filler.

56. The disposable absorbent article of claim 46, wherein at least a portion of the outer cover is activated.

57. The disposable absorbent article of claim 46, further comprising one or more waist bands and one or more leg bands joined to at least a portion of the backsheet, wherein the waist bands and leg bands substantially encircle the waist and legs, respectively, of a wearer when the wearable absorbent article is worn by the wearer.
